(19) United States
(12) Patent Application Publication
(10) Pub. No.: US 2015/0132393 A1
(43) Pub. Date: May 14, 2015

(54) NANOSTRUCTURE COATED WITH A TWIST-STRAINED DOUBLE-STRANDED CIRCULAR DEOXYRIBONUCLEIC|ACID (DNA), METHOD FOR MAKING AND USE

(71) Applicant: Sonia TRIGUEROS, Oxford (GB)

(72) Inventor: Sonia Trigueros, Oxford (GB)

(21) Appl. No.: 14/397,834

(22) PCT Filed: Apr. 26, 2013
(86) PCT No.: PCT/GB2013/051069

§ 371 Date: Oct. 29, 2014

(30) Foreign Application Priority Data
Apr. 30, 2012 (GB) 1207484.5

(51) Int. Cl. A61K 47/26; A61K 47/02; A61K 9/14

(52) U.S. Cl. CPC A61K 47/26; A61K 9/14; A61K 47/02
USPC 424/490; 427/2.12; 428/403

(57) ABSTRACT

A SWCNT coated with a twist-strained double-stranded circular deoxyribonucleic acid (DNA) is provided, together with methods for preparing the coated SWCNT, for removing the DNA coating from the SWCNT, and for sorting SWCNTs using the DNA coating.

### FIELD OF THE INVENTION

The invention relates to a means for manipulation, storage and sorting of nanostructures. The invention relates to nanostructures as products, where the nanostructure is coated with a twist-strained double-stranded circular deoxyribonucleic acid (DNA). The invention also relates to methods for preparing such products and methods for removing the DNA coating from the products. The invention further relates to methods for sorting nanostructures using the DNA coating.

### BACKGROUND TO THE INVENTION

Carbon nanostructures and nanostructures made from other materials are materials with unusual properties having a variety of applications in the field of nanotechnology including in electronics, optics, and medicine. Manipulation of nanostructures, such as carbon nanotubes, is made difficult due to their instability and tendency to aggregate forming macro-aggregates. This presents significant difficulties for manufacture of carbon nanostructures on a commercial scale and complicates the provision of carbon nanostructures in soluble form for applications where this is necessary. Another problem is difficulty in sorting of carbon nanostructures by size or other parameters, where a subset of nanostructures having specific features is required for a particular application. Production methods known in the art do not allow for the specific manufacture of carbon nanostructures of a particular type, and as such the nanostructures need to be separated according to the desired type.

Separation of carbon nanostructures, such as carbon nanotubes, by use of single stranded DNA has been described previously. This method relies on relatively strong pi-stacking between aromatic DNA base units and the nanotube surface. This method is non-reversible. Also, this method does not have more general applicability to non-carbon nanostructures.

There is a need for provision of further means for manipulation, storage and sorting of nanostructures.

### SUMMARY OF THE INVENTION

The present invention utilises a nanostructure coated with a twist-strained double-stranded circular DNA. It has been surprisingly found that inducing a topological change in relaxed circular double stranded DNA to a twist-strained form in the presence of nanostructures coats the nanostructures with the DNA. Furthermore, the coated nanostructures thus formed are soluble and stable in physiological mediums. The coating of the nanostructures also makes them amenable to sorting, for example based on the size of the nanostructures. Additionally, the coating is readily reversible, allowing the nanostructures to be released after storage or sorting. The reversibility of the coating is achieved by altering conditions such that there is a change in topology in the DNA from a twist-strained to a relaxed form, which releases the coating from the nanostructure. The twist-strained DNA does not directly bind to the surface of the nanostructure, but is wrapped around the structure. The coating is not therefore dependent on the particular material used in the nanostructure.

The invention thus provides a nanostructure coated with a twist-strained double-stranded circular deoxyribonucleic acid (DNA). The invention further provides a composition comprising a nanostructure in solution, wherein said composition comprises conditions promoting coating of said nanostructure with a twist-strained double-stranded circular DNA. The invention additionally provides a method for coating a nanostructure with a twist-strained double-stranded circular DNA, comprising incubating a nanostructure with a relaxed double-stranded circular DNA under conditions promoting a change in topology of said DNA to twist-strained double-stranded circular DNA, to thereby coat the nanostructure.

The invention also provides a method for removing a twist-strained double-stranded circular DNA from a nanostructure, which comprises changing incubation conditions to promote removal of said DNA. The invention further provides a method for sorting nanostructures by size, comprising separating nanostructures that are coated with twist-strained double-stranded circular DNA.

The invention additionally provides a nanostructure coated with a twist-strained double-stranded circular DNA for use in a method for treatment of the human or animal body by surgery or therapy or a diagnostic method practised on the human or animal body. The nanostructure coated with a twist-strained double-stranded circular DNA may be for use in a method for delivering a diagnostic or therapeutic agent. The invention further provides a method for delivering a therapeutic agent to a subject in need thereof comprising administering an effective amount of said therapeutic agent by delivery of a nanostructure coated with a double-stranded circular DNA.

### BRIEF DESCRIPTION OF THE FIGURES

FIG. 1 shows in panel A the removal of supercoiling from DNA using vaccinia virus Topoisomerase I (Type 1B). 0.7% agarose gel electophoresis shows native supercoiled DNA (sigma superhelical density=0.06) and relaxed DNA produced by Topo 1B (sigma superhelical density=0). Panel B shows the formation of a coated carbon nanotube by DNA wrapping in the presence of MgCl₂.

FIG. 2 top half shows a schematic for filter binding purification of coated carbon nanostructures. Panel A shows a microcentrifuge tube; centrifugation is used to pellet uncoated carbon nanostructures as shown by arrow. Free DNA and coated carbon nanostructures remain soluble. Panel B shows the introduction of a nitrocellulose filter into the microcentrifuge tube and addition of a sample to the top of the filter. The sample contains free DNA and coated carbon nanostructures. Panel C shows free DNA passes through the filter to the bottom of the tube (open circles) while coated carbon nanostructures are recovered from the top of the filter.

FIG. 2 bottom half shows the results of a filter binding purification method carried out per the schematic (samples on lanes 1, 2, 3 and 4: double stranded DNA+100 mM MgCl₂; samples on lanes 5,6 and 7: double stranded DNA+swcnts+100 mM MgCl₂; samples on lanes 8,9 and 10: double stranded DNA+swcnts in H₂O). Lane 1=double stranded DNA control. Lane 2=coating reaction product (double stranded DNA) at 100 mM MgCl₂ before filter binding purification method. Lane3=double stranded DNA recovered after washing filter with 100 microlitres of 100 mM MgCl₂. Lane 4=double stranded DNA eluted from the top of the filter. Lane 5=coating reaction product (double stranded DNA wrapped on nanotubes) in 100 mM MgCl₂ before filter binding purification method. Lane 6=free double stranded DNA recovered after washing filter with 100 microlitres of 100 mM MgCl₂. Lane 7=double stranded DNA eluted from the top of the filter. Lane 8=coating reaction product (double stranded DNA and nanotubes) in H₂O before filter binding purification method. Lane 9=double stranded DNA recovered after washing filter with 100 microlitres of H₂O. Lane 10=double stranded DNA eluted from the top of the filter.

FIG. 3 shows atomic force microscopy images. Panel A=relaxed and supercoiled DNA dried on a mica surface in H₂O, Scale is shown in nanometers. Panel B=relaxed DNA dried on a mica surface in 100 mM MgCl₂. Scale is shown in micrometers. Panel C: single wall carbon nanotubes (SWNTs) in H₂O. Inset shows dry AFM height measurement in nm of a newly synthesized nanotube. Panel D: aggregated single wall carbon nanotubes 5 minutes after synthesis. Scale is shown in micrometers. Panel E: coated carbon nanotubes produced in accordance with the method of the invention. Scale is shown in micrometers. Panel F: detailed image of the coated carbon nanotubes of Panel D.

FIG. 4 shows atomic force microscopy images illustrating the DNA unfolding from coated carbon nanotubes under various conditions. Panel A=uncoated SWNTs (top arrow) and free DNA (bottom arrow) in 15 mM MgCl₂. Scale is shown in micrometers. Panel B=uncoated SWNTs (bottom arrow) and free DNA (top arrow) in 30 mM MgCl₂. Scale is shown in micrometers. Panel C=uncoated SWNTs (bottom arrow) and free DNA (top arrow) following incubation for 20 minutes at 45 degrees centigrade. Scale is shown in micrometers. Panel D=detailed image from Panel A. Scale is shown in nanometers. Panel E=detailed image from Panel B. y axis: micrometers, x axis: nanometers. Panel F=detailed image from Panel C. Scale is shown in nanometers.

FIG. 5 shows atomic force microscopy images of coated carbon nanotubes stored under various conditions. All scales are shown in micrometers. Panel A: SWCNTs/DNA incubated at 30° C. for 3 months in Fetal Bovine Serum. Panel B: SWCNTs/DNA incubated at 30° C. for 3 months in Bacteria growth medium LB. Panel C: SWCNTs/DNA incubated at 30° C. for 3 months in Yeast growth Medium YPD. Panel D SWCNTs/DNA incubated at 30° C. for 3 months in 100 mM MgCl₂. Panel E: SWCNTs/DNA incubated at 37° C. for 3 months in 100 mM MgCl₂. Panel F: SWCNTs/DNA incubated at 4° C. for 3 months in 100 mM MgCl₂.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting. In addition as used in this specification and the appended claims, the singular forms “a”, “an”, and “the” include plural referents unless the content clearly dictates otherwise. Thus, for example, reference to “a compound” includes “compounds”, reference to “a polypeptide” includes two or more such polypeptides, and the like. All publications, patents and patent applications cited herein, whether supra or infra, are hereby incorporated by reference in their entirety.

### Coated Nanostructures

The invention provides a nanostructure coated with a twist-strained double-stranded circular DNA.

Nanostructures

A nanostructure is a structure having one, two or three dimensions on the nanoscale. A dimension on the nanoscale is typically between 0.1 nm and 100 nm. A nanosurface, such as a nanotextured surface typically has only one spatial dimension on the nanoscale. Preferably, the thickness of the nanosurface is on the nanoscale. A spherical nanoparticle may have all three dimensions on the nanoscale. Any suitable nanostructure and nanoparticle can be used. In particular, the coating method of the present invention does not rely on a direct interaction with the surface nanoparticle or nanostructure. The twist-strained double-stranded circular DNA essentially wraps around the nanostructure or nanoparticle as it is formed.

Suitable nanostructures and nanoparticles include the following: carbon based carriers such as fullerenes, carbon nanotubes including both single wall and multiple wall carbon nanotubes, quantum dots of cadmium and/or zinc selenides, dendrimers, ceramic-matrix nanocomposites, metal-matrix nanocomposites, polymer-matrix nanocomposites, nanobones, nanorods, nanoshells, nanospheres, nanocapsules, nanopores, magnetic nanoparticles, metal nanoparticles and semi conductors such as gold nanoparticles, silver nanoparticles, copper nanoparticles and chromium nanoparticles, bimetallic nanoparticles, lipoplexes and polyplexes and liposomes.

The nanostructure may be functionalised by including functional groups at its surface. The nanostructure may include positive or negative functional groups at its surface. The nanostructure may have a net positive or net negative charge. The nanostructure may be uncharged.

In other embodiments, the nanostructure is not functionalised, or does not include polar or positive functional groups at its surface. The nanostructure may not include any polyethylene glycol (PEG) group. The nanostructure may not include any ammonium groups, lysine groups, or amino groups. Where the nanostructure does include functional groups, such as positive functional groups at its surface, the functional groups are not required for the wrapping of DNA around the nanostructure. In other words, the nanostructure may be coated with twist-strained double-stranded circular DNA without interaction between the functional groups on the nanostructure and relaxed double-stranded DNA.

In a preferred aspect of the present invention, the nanostructure is a carbon nanostructure comprising carbon which has at least one dimension on the nanoscale. The carbon nanostructure may comprise one or more other substances in addition to carbon, such as a ceramic, a metal or other inorganic material. The carbon nanostructure may thus be a nanocomposite of carbon and other materials. A carbon nanostructure may have any shape. A carbon nanostructure may be a nanoparticle, a nanosphere, a nanosurface, a fullerene or a nanotube. A carbon nanostructure may be a planar surface or may be spherical or cylindrical.

In some embodiments, the carbon nanostructure is not a cationic fullerene. Preferably, the carbon nanostructure is a nanotube, preferably a carbon nanotube. A carbon nanotube is an allotrope of carbon having a cylindrical nanostructure. Typically a nanotube is hollow, but the invention may also utilise nanotubes such as carbon nanotubes having components present inside and/or outside the cylindrical nanostructure. Such components may include other carbon nanostructures such as fullerenes. An example is a carbon peapod.

It should be understood that in addition to having a tubular structure, a nanotube may also comprise other nanostructures. Thus, a carbon nanotube may be capped at one or both ends by, or have present at any other location, a spherical or hemispherical nanostructure, such as a fullerene. The fullerene may be a buckyball. The carbon nanotube may be a nanobud. The carbon nanotube may comprise graphene sheets or leaves, such as graphitic foliates attached to a surface of the nanotube. The carbon nanotube may be a graphenated carbon nanotube (g-CNT).

The nanotube such as a carbon nanotube is typically in an elongated tubular conformation. However, a nanotube having any conformation may be used. An example is a nanotorus.

The nanotube such as a carbon nanotube may be single-walled, thus typically comprising or consisting of a one atom thick cylindrical layer of carbon. A Single-Walled Carbon NanoTube is commonly abbreviated as a SWNT or SWCNT. The nanotube, such as a carbon nanotube may be multi-walled, thus comprising or consisting of multiple cylindrical layers of carbon. A Multi-Walled Carbon NanoTube is commonly abbreviated as a MWNT. A MWNT may comprise two, three, four or more cylindrical layers of carbon. Each cylindrical layer of carbon in a MWNT is typically a one atom thick cylindrical layer of carbon. In some embodiments, the carbon nanostructure is not a MWNT.

The nanotube may be of any length or diameter. These parameters will typically be selected according to the applications for which the nanotube is to be used.

The nanotube can be about 10 micrometers in length, although longer nanotubes may also be used. The nanotube can be 2 nanometers or greater in length, preferably 5 nanometers or greater in length. Preferably, the nanotube is about 1 micrometer or less in length, such as about 700 nanometers or less in length. The nanotube may thus be from about 2 nanometers to about 10 micrometers in length. Preferably, the nanotube has a length in the range of about 5 nanometers to about 700 nanometers. The nanotube may have a length in the range of about 10 to about 700, such as about 20 to about 700, about 50 to about 700, about 100 to about 700, about 100 to about 500, about 150 to about 500, about 200 to about 500, about 300 to about 500, about 100 to about 400, about 100 to about 300, about 100 to about 200, or about 300 to about 400 nm in length. Nanotubes in the length range of about 100 nm to about 700 nm are particularly preferred in applications where they are required to be soluble. In a preferred embodiment, the nanotube is a carbon nanotube having the dimensions referred to above.

The nanotube is typically at least about 400 picometers in diameter or greater. The nanotube is typically less than 100 nanometers in diameter, although wider nanotubes may also be used. Where SWCNT are used, the diameter of the nanotube is typically about 400 picometers to about 2 nanometers, such as about 400 picometers to about 500 picometers, about 400 picometers to about 1 nanometer, about 500 picometers to about 1.5 nanometers or about 500 picometers to about 1 nanometer.

A nanostructure may be provided as part of a mixture of nanostructures having different dimensions. For example, a carbon nanotube may be provided as part of a mixture of carbon nanotubes having any length or diameter, or as a mixture of carbon nanotubes having a specific length range or diameter range. Examples of preferred length and diameter ranges are described above.

The carbon nanostructure may be functionalised by including functional groups at its surface. The carbon nanostructure may include positive or negative functional groups at its surface. The carbon nanostructure may have a net positive or net negative charge. The carbon nanostructure may be uncharged. In other embodiments, the carbon nanostructure is not functionalised, or does not include polar or positive functional groups at its surface. The carbon nanostructure may not include any ammonium groups, lysine groups, or amino groups. Where the carbon nanostructure does include functional groups, such as positive functional groups at its surface, the functional groups are not required for the wrapping of DNA around the carbon nanostructure. In other words, the carbon nanostructure may be coated with twist-strained double-stranded circular DNA without interaction between the functional groups on the carbon nanostructure and relaxed double-stranded DNA.

Double-Stranded Circular DNA

The DNA coated on the carbon nanostructure is a double-stranded circular DNA. The DNA may be of any sequence, of any length and of any origin. The DNA may be a plasmid or any other form of circular DNA. The DNA is typically present wrapped around the outer surface of the nanostructure. The nanostructure may be coated with one or more double-stranded circular DNAs. The DNA is typically non-covalently associated with the nanostructure. The DNA is typically not conjugated to the nanostructure, such as the carbon nanostructure. The DNA typically does not bind to or electrostatically interact with polar or positively charged functional groups on the nanostructure.

The DNA coated on the nanotube is in twist-strained form. The DNA may be overtwisted. The DNA may be present in supercoiled form. A twist-strained DNA typically has an increased number of helical turns as compared to the relaxed form of the same DNA.

A twist-strained DNA is characterised by having a linking number Lk which differs from Lk0, the linking number observed in a torsionally relaxed circular double-stranded form of the same DNA. Lk is related to two geometrical parameters of the DNA double helix, the helical twist (Tw) and the contortion of the DNA axis, known as the writhe (Wr). The linking number Lk is the sum of Tw+Wr. Lk0 is calculated by the number of base pairs present in the circular double-stranded DNA divided by the helical repeat, which is about every 10.5 base pairs.

The linking number difference observed between a twist-strained DNA and a relaxed DNA is termed deltaLk. deltaLk is Lk−Lk0, and is typically expressed as the superhelical density sigma using the equation: sigma=deltaLk/Lk0. A twist-strained DNA typically has a superhelical density (sigma) value of greater than 0. A twist-strained DNA may have less than 10 base pairs per turn of the double helix. A twist-strained DNA may have a writhe value different than 0. Where Lk deviates from Lk0, the increased torsional stress within the helix is typically converted into an decrease of writhe value, thereby forming a supercoiled structure.

Further Components

The coated nanostructure of the invention, such as a carbon nanotube may comprise further components. For example, a coated nanotube may comprise further components inside the cylindrical nanostructure, on the surface of the nanotube, or associated with the coated DNA.

The coated nanostructure may comprise cations. Any suitable cation or a combination of different cations may be present. Preferably, the coated nanostructure comprises divalent cations, such as Mg²⁺. The inclusion of cations assists in producing twist-strained DNA. The inclusion of cations may also assist in coating of the DNA on the nanotube by neutralising the negatively charged DNA backbone and also any negative charge present on the nanotube. However, the wrapping of DNA around the nanostructure occurs through a change in DNA topology. The coated nanostructure may further comprise an anion as a counterion to the cation. Any suitable anion may be present. The anion may be a monovalent or divalent anion. An example is Cl⁻.

The coated nanostructure may comprise any biologically or therapeutically active agent such as a chemotherapeutic agent or an antibiotic, as described in more detail below in relation to medical applications of the invention. The coated nanostructure may comprise any other components suitable for applications in electronics. Such components may comprise a carbon-nanotube polymer composite or a metal coating, such as a copper or aluminium coating. The coated carbon nanostructure, such as a coated carbon nanotube, may comprise stored hydrogen.

### DNA Coating

The invention additionally provides a method for coating a nanostructure with a twist-strained double-stranded circular DNA. The method comprises incubating a nanostructure with a relaxed double-stranded circular DNA under conditions promoting coating of said nanostructure with a twist-strained double-stranded circular DNA.

It has been surprisingly found that double-stranded circular DNA may be used to coat nanostructures by incubating double stranded circular DNA having a relaxed topology under conditions which promote a change in topology to a twist-strained form.

A relaxed double-stranded circular DNA typically does not have any supercoiling, such as positive or negative supercoiling. A relaxed DNA has an Lk0 as described above. A relaxed DNA typically has a superhelical density (sigma) value of about 0. A relaxed DNA typically has a writhe value of about 0. A relaxed DNA typically has about 10.5 base pairs per turn of the double helix. A relaxed DNA may have an average sigma value of about 0, but include some regions of supercoiling.

A relaxed double stranded circular DNA may be obtained in a number of ways. A double stranded circular DNA may be obtained commercially in relaxed form. Alternatively, a double stranded DNA may be generated in relaxed form from a positively or negatively supercoiled DNA. Typically, a double-stranded circular DNA obtained from a prokaryote or eukaryote will be supercoiled. For example, a double-stranded plasmid isolated from a bacterium such as E. Coli will typically have an average negative superhelical density. Supercoiled double-stranded DNA may be converted into relaxed DNA for use in coating according to the invention by incubation with a topoisomerase enzyme. Any suitable Type IB topoisomerase enzyme of any origin may be used. An example of a suitable topoisomerase enzyme is Topoisomerase I from vaccinia virus (obtainable commercially as Epicentre catalogue number VT710500). Suitable conditions for generation of relaxed DNA using this enzyme are described below.

The relaxed DNA is incubated with the nanostructure to be coated as discussed below. Prior to this incubation, the method may comprise a step of pre-incubation of the nanostructures with cations. This step is preferably used to neutralise any charge present on the nanostructure and thereby facilitate coating with DNA. Suitable cations are described below. The cations may be the same cations used to promote coating of the nanostructure with twist-strained DNA or may be different cations. The cations are typically present at a higher concentration for charge neutralisation than that is used to promote coating with DNA. A suitable cation concentration for charge neutralisation may be from about 50 mM to about 100 mM, more preferably about 100 mM.

Subsequent to any pre-incubation of the carbon nanostructures as described above, the relaxed DNA is incubated with a nanostructure under conditions promoting coating of said nanostructure with twist-strained DNA. The conditions promote a change in topology in the DNA from a relaxed form to a twist-strained form coated or wrapped around the nanostructure. The conditions may both promote coating of and stabilise coating of nanostructures with twist-strained double stranded circular DNA.

The conditions typically promote coating of at least 30%, more preferably at least 40%, 50%, 60%, 65%, 70%, 80%, 90%, 95% or more of the nanostructures present in the composition with twist-strained DNA. The conditions may promote coating of all of the nanostructures present in the composition with twist-strained DNA. The skilled person can select a suitable time period empirically for incubation of the nanostructure with the DNA to generate a desired yield of coated nanostructures.

The conditions promoting coating of the nanostructure with twist-strained DNA typically comprise the presence of cations. Cations can cause a change in topology from relaxed to twist-strained DNA, thereby coating the nanostructure. The coating of the nanostructure is dependent on the change of topology of DNA, and not simply on chelation of the DNA and the nanostructure by the cations. Cations can also neutralise charge present on the nanostructures, thereby facilitating the coating with DNA.

Any suitable divalent cation or a combination of different divalent cations may be present. Preferably, divalent cations are present, such as Mn²⁺, Ca²⁺, Co²⁺ or Mg²⁺. The inventor has found that these cations have a high effect on overwinding the helical repeat of the double helix, thereby being particularly suitable for coating the nanostructure with twist-strained DNA. The above divalent cations respectively caused the double helix to overwind by 0.48 degrees per base pair, 0.49 degrees per base pair, 0.44 degrees per base pair and 0.40 degrees per base pair, when incubated at 0 to 4 degrees centigrade. Preferably, the divalent cation is Mg²⁺.

The cations are typically associated with the DNA and/or the nanostructure. Cations may also be present in solution. The solution may further comprise anions as counterions to the cations. Any suitable anion may be present. The anion may be a monovalent or divalent anion. An example is Cl⁻. The solution may thus comprise a salt of the cation and anion in solution, such as MgCl₂.

Any cations are present at a concentration which promotes coating of nanostructures with twist-strained double stranded circular DNA. The skilled person is able to select a suitable working concentration of cations empirically, depending on the particular cation to be used. A suitable concentration of a divalent cation for coating is typically greater than about 40 mM, such as at least about 45 mM, 50 mM, 55 mM or 60 mM. More preferably the concentration is about 65 mM, about 70 mM, about 75 mM, or about 80 mM. Typically, the concentration of the divalent cation is in the range of 55 mM to 80 mM, such as 60 mM to 80 mM, 65 mM to 80 mM, 70 mM to 80 mM, 50 mM to 70 mM, 55 mM to 70 mM, 60 mM to 70 mM. The concentration of the divalent cation is typically less than about 85 mM.

The above minimum concentrations and concentration ranges are particularly preferable where the divalent cation is Mg²⁺, and where Mg²⁺ is provided in the form of MgCl₂. Thus, for example, Mg²⁺ may be provided at a concentration of at least about 70 mM, or in the range of 60 mM to 80 mM. Preferably, the conditions for coating comprise a MgCl₂ concentration of at least about 70 mM or in the range of at least about 60 mM to about 80 mM.

The conditions promoting coating may comprise the presence of both divalent and monovalent cations. Preferably, the conditions comprise the presence of Mg²⁺ and Na⁺. Both of these cations may be present in combination with Cl⁻ as a counterion. The conditions may comprise Mg²⁺ at a concentration of at least about 70 mM, or in the range of 60 mM to 80 mM and Na⁺ at a concentration of about 15 mM, or in the range of 10 mM to 25 mM. The conditions may comprise a MgCl₂ concentration of at least about 70 mM and an NaCl concentration of about 15 mM. The use of NaCl at a concentration of about 15 mM is preferred where the nanostructures are to be used for a biological or medical application since this approximates physiological salt concentrations.

The conditions promoting coating preferably comprise a temperature in the range of 0 to 10 degrees centigrade. The temperature is typically not greater than about 35 degrees centigrade or about 30 degrees centigrade, preferably less than about 25 degrees centigrade. The temperature may be at least about 0 degrees centigrade to about room temperature. The temperature may be at least about 0 degrees centigrade to about 25, about 20, about 18, about 15, about 10, about 8 or about 4 degrees centigrade. The temperature may be about 0, about 4, about 8, about 10, about 15, about 20 or about 25 degrees centigrade.

The conditions promoting coating may comprise a pH in the range of about 6 to about 10, although higher or lower pH values may also be employed. The conditions may comprise a neutral or physiological pH, such as a pH of about 7.5.

The method of DNA coating of the invention may be carried out using any suitable working concentration of nanostructures. The working concentration selected by the skilled person may depend for example on the dimensions of the nanostructures, such as on the size and diameter of carbon nanotubes. An example of a suitable concentration of carbon nanotubes is about 50 to about 100 μg/ml, such as about 70 μg/ml. These may be the concentrations of uncoated carbon nanotubes or of coated carbon nanotubes. Uncoated carbon nanotubes may be present in a weight-to-volume ratio of about 200 to about 1, about 100 to about 1, about 90 to about 1, about 80 to about 1, about 70 to about 1, about 60 to about 1, about 50 to about 1 with free double stranded circular DNA.

The method of DNA coating of the invention may be carried out using any suitable working concentration of double stranded circular DNA. The working concentration selected by the skilled person may depend for example on the size of the double stranded circular DNA. At least some of the double stranded circular DNA is present in relaxed form, but supercoiled forms of DNA may also be present. At least 50%, 60%, 70%, 80%, 90%, 95% or all of the double stranded circular DNA used for coating may be present in relaxed form.

An example of a suitable concentration of double stranded circular DNA is about 0.5 to about 10 μg/ml, such as about 1 μg/ml. Suitable concentrations may vary depending on the size of the double-stranded circular DNA, and may be determined empirically by the skilled person. Optimal coating may be obtained using a concentration of 10 nanograms/microliter for a 1.5 kb DNA, 5 nanograms/microliter for a 3 kb DNA and 2.5 nanograms/microliter for a 6 kb DNA. The double stranded circular DNA may be present in a weight-to-volume ratio of about 0.5 to about 100, about 1 to about 100, about 1.2 to about 100, about 1.5 to about 100, about 2 to about 100, or about 2.5 to about 100 with uncoated carbon nanostructures, such as uncoated carbon nanotubes.

The above concentrations and weight-to-volume ratios may be those of relaxed double stranded circular DNA. The above concentrations and weight-to-volume ratios are those of free DNA in solution.

The method may be carried out to coat any form of nanostructure described herein with twist-strained double-stranded circular DNA. The method is preferably carried out to coat carbon nanotubes. The method is particularly preferably carried out to coat SWCNTs which have a length in the range of about 100 to about 700 nanometers, such as about 100 to about 600, about 100 to about 500, about 150 to about 500, about 200 to about 500, about 300 to about 500, about 100 to about 400, about 100 to about 300, about 100 to about 200, or about 300 to about 400 nm in length.

SWCNTs having lengths in these ranges may be prepared by incubation of longer carbon nanotubes under acidic conditions. The invention thus provides a method for producing carbon nanotubes which have a length in the range of about 100 to about 700 nm and which are coated with twist-strained double-stranded circular DNA. The method comprises incubating carbon nanotubes of at least about 1 micrometer in length under acidic conditions promoting production of carbon nanotubes having a length in the range of about 100 to about 700 nm. The method further comprises incubating a thus produced carbon nanotube with relaxed double-stranded circular DNA under conditions promoting coating of said carbon nanotube with twist-strained double-stranded circular DNA. The carbon nanotubes are preferably SWCNTs.

The acidic conditions promoting production of carbon nanotubes having a length in the range of about 100 to about 700 nm typically comprise incubation with a mixture of concentrated sulphuric acid and nitric acid. The concentrated sulphuric acid may have a mass fraction of at least 98%, preferably 99.999% H₂SO₄. The nitric acid may have a mass fraction of at least 70% or more HNO₃. The sulphuric acid may be present in a ratio of about 3 to about 1 with the nitric acid. The incubation is carried out for sufficient time to generate carbon nanotubes in the desired length range. The skilled person is able to select a suitable time period empirically by analysis of the length range of the carbon nanotubes produced after particular time periods. Suitable time periods for generation of carbon nanotubes in various length ranges starting from carbon nanotubes in the 1 to 10 micrometer length range are described below.

The incubation is typically carried out at a temperature of at least 60 degrees centigrade or higher. The incubation may comprise sonication of the carbon nanotubes.

The acid digested carbon nanotubes thus produced typically have a net negative charge, having carboxylic acid groups at their surfaces and open ends.

The acid digested carbon nanotubes are typically processed prior to coating with relaxed double-stranded DNA to remove excess acid and side-products, and to provide the carbon nanotubes under conditions promoting their coating with twist-strained double-stranded circular DNA. Suitable processing steps may be provided by the skilled person based on their common general knowledge. The processing may comprise filtration, such as vacuum filtration of the carbon nanotubes. The processing may comprise washing of the carbon nanotubes under suitable conditions to return their pH to about neutral pH, such as by washing a filter coated with the carbon nanotubes. The processing may comprise sonication to remove the carbon nanotubes from a filter. The processing may further comprise precipitation of the carbon nanotubes by centrifugation. The processing may further comprise formulation of the carbon nanotubes under conditions promoting coating with twist-strained double-stranded circular DNA as described above.

The thus processed carbon nanotubes may then be coated with twist-strained double-stranded circular DNA by a method of coating as described above.

A method of coating of the invention may further comprise separating nanostructures coated with twist-strained double-stranded circular DNA from free DNA and/or uncoated nanostructures. This separation may be carried out by any suitable means.

The separation of coated nanostructures from uncoated nanostructures may be carried out by centrifugation to precipitate the insoluble uncoated nanostructures. The soluble coated nanostructures remain in the supernatant.

The separation of coated nanostructures from free DNA may be carried out by filtration. The filtration may be carried out using a nitrocellulose membrane. Preferably, the nitrocellulose membrane has a binding capacity in the range of 80 to 150 micrograms per square centimetre. The nitrocellulose membrane typically has a pore size of 0.45 micrometers or greater. The coated nanostructures can bind to the filter used, such as the nitrocellulose membrane whereas free DNA is not retained on the filter.

The present invention also relates to a method for storing nanostructures coated with twist-strained double-stranded circular DNA under conditions stabilising coating of nanostructures with twist-strained double stranded circular DNA. Suitable stabilising conditions include the conditions described above for promoting coating of carbon nanostructures with twist-strained double stranded circular DNA.

The stabilising conditions typically comprise a temperature of less than 40 degrees centigrade; preferably the storage is carried out at about 0 to about 10 degrees centigrade. Preferably, the storage is carried out at a pH of 5 to 12. The coated nanostructures may be frozen and stored at a temperature in the range of about minus 120 degrees centigrade to about 0 degrees centigrade. The coated nanostructures may be frozen and lyophilised. Lyophilised coated nanostructures may then be reformulated. The reformulation may be performed under the same conditions used for stabilising and coating as described above. The stabilising conditions may be used to store nanostructures such as carbon nanostructures coated with twist-strained double stranded circular DNA for at least one week, at least two weeks, at least one month, at least two months, at least three months, at least six months or more, without significant aggregation of the nanostructures.

The coated nanostructures may be stored in a physiological medium such as bacteria growth medium, a yeast growth medium or serum. This is of particular use where the nanostructures are to be used for medical applications. The bacteria growth medium may be Luria Bertani medium. The yeast growth medium may be may be YEPD medium comprising yeast extract, peptone and dextrose. The serum may be fetal bovine serum.

The coated nanostructures, such as carbon nanostructures may be stored in an organic solvent. Suitable solvents include cyclohexyl-pyrrolidone (CHP) and 1-benzyl-2-pyrrolidinone (NBenP), dimethylformamide (DMF), N-methylpyrrolidone (NMP), hexamethylphosphoramide (HMPA), monochlorobenzene (MCB), ortho-dichlorobenzene (o-DCB), meta-dichlorobenzene (m-DCB) and 1, 2, 4-trichlorobenzene (TCB). Storage in an organic solvent is of particular use where the nanostructures are to be used for applications in electronics.

The invention further provides a composition comprising a nanostructure, and in particular a carbon nanostructure in solution, wherein said composition comprises conditions promoting coating of said nanostructure with twist-strained double stranded circular DNA. The conditions may both promote coating of and stabilise coating of nanostructures with twist-strained double stranded circular DNA. The composition may be obtained or obtainable by a method of coating of the invention.

The composition may be a mixture of uncoated nanostructures and free double stranded circular DNA. The composition may be a mixture of nanostructures coated with twist-strained double stranded circular DNA, free DNA and uncoated nanostructures. Alternatively, the only nanostructures present in the composition may be nanostructures, which are coated with twist-strained double stranded circular DNA.

At least 30% of the nanostructures present in the composition may be coated with twist-strained double stranded circular DNA. More preferably at least 40%, 50%, 60%, 70%, 80%, 90%, 95% or all of the nanostructures present in the composition may be coated with twist-strained double stranded circular DNA.

Where the composition comprises coated carbon nanostructures, and for example has been prepared by a method of coating of the invention, there may be no free DNA remaining in solution. Alternatively, about 50% or less, more preferably about 40%, about 30%, about 20%, about 10%, about 5%, or less of the DNA present in the composition may be present as free DNA.

Preferably, the composition comprises a MgCl₂ concentration in the range of at least about 60 mM to about 80 mM, and has a pH of about 6 to about 10 and a temperature of about 0 degrees centigrade to about 10 degrees centigrade. This composition may comprise at least 60% of the carbon nanostructures present in the composition coated with twist-strained double-stranded circular DNA, and in solution.

Uncoating

It may be desirable to remove a twist-strained double-stranded circular DNA from a nanostructure. For example, as discussed below, the twist-strained double-stranded circular DNA may be utilised to stabilise, store or sort nanostructures, but may then no longer be needed. Surprisingly, the coating of the nanostructures with a twist-strained double-stranded circular DNA has been found to be readily reversible. The invention provides a method for removing a twist-strained double-stranded circular DNA from a nanostructure, comprising changing incubation conditions to promote removal of said DNA. The method thus comprises incubating a nanostructure coated with a twist-strained double-stranded circular DNA under conditions promoting removal of said DNA.

The change in incubation conditions typically promotes relaxation of the twist-strained DNA. The method may therefore be used to relax a twist-strained double-stranded DNA coated on a nanostructure.

The change in incubation conditions to promote removal of the DNA may comprise reducing the cation concentration. Thus, the cation concentration is reduced as compared to the concentration used for coating of the nanostructures with twist-strained double stranded circular DNA. The concentration of divalent cations may be reduced. The conditions promoting removal of twist-strained double-stranded circular DNA may comprise a divalent cation concentration of about 30 mM or lower. The binding of the double-stranded circular DNA to the nanostructure is typically unstable under these conditions. The conditions may comprise a divalent cation concentration of about 25 mM or lower, about 20 mM or lower, about 15 mM or lower or about 10 mM or lower. The conditions may comprise the absence of any divalent cations. Typically, the conditions comprise Mg²⁺ concentration or about 30 mM or lower, such as about 25 mM or lower, about 20 mM or lower, about 15 mM or lower or about 10 mM or lower.

Such conditions may be imposed by any suitable means of changing the concentration of cations in a solution comprising the coated carbon nanostructures of the invention. For example, the solution may be dialysed against a solution having a divalent cation concentration of 30 mM or lower as described above. The solution may be diluted to reduce the concentration of cations. A chelating agent may be added to reduce the concentration of cations. The change in incubation conditions to promote removal of the DNA may comprise an increase in temperature. Thus, the temperature is increased as compared to the temperature used for coating of the carbon nanostructures with twist-strained double stranded circular DNA. The conditions promoting removal of twist-strained double-stranded circular DNA may comprise a temperature of about 45 degrees centigrade or higher. The temperature may be about 50, about 55, about 60, about 70, about 80 or about 90 degrees centigrade or higher. The temperature may be in the range of about 45 to about 60 degrees centigrade. Typically, the temperature is less than 100 degrees centigrade.

Method for Sorting

The invention permits the sorting of nanostructures by size, through the solubilisation of nanostructures such as carbon nanostructures by coating with a twist-strained double-stranded circular DNA. The soluble coated nanostructures may be separated by size on the basis of their DNA coating using any suitable DNA separation technique. Such techniques include DNA electrophoresis, size-exclusion chromatography, silica adsorption and density gradient centrifugation. The DNA may be separated in a micro-channel by silica adsorption. The DNA electrophoresis may be carried out on an agarose or polyacrylamide gel.

Preferably, the method is used to sort carbon nanotubes, more preferably to specifically sort carbon nanotubes which are 100-700 nanometers in length from carbon nanotubes of other lengths.

Medical Applications

The coated nanostructures of the invention, such as the coated carbon nanostructures, are particularly suitable for medical applications since they are soluble in physiological media.

The invention thus provides a nanostructure coated with a twist-strained double-stranded circular DNA for use in a method for treatment of the human or animal body by surgery or therapy or a diagnostic method practised on the human or animal body. The invention additionally provides a nanostructure coated with a twist-strained double-stranded circular DNA for use in a method for delivering a diagnostic or therapeutic agent.

The invention further provides use of a nanostructure coated with a twist-strained double-stranded circular DNA in the manufacture of a medicament for delivering a therapeutic or diagnostic agent. The invention also provides a method for delivering a therapeutic agent to a subject in need thereof comprising administering an effective amount of said therapeutic agent by delivery of a nanostructure coated with a double-stranded circular DNA.

In the above medical applications, a therapeutic or diagnostic agent may be covalently or non-covalently attached to the coated nanostructure, such as a coated carbon nanostructure. The therapeutic or diagnostic agent may be covalently or non-covalently bonded to the twist-strained double-stranded circular DNA. The therapeutic or diagnostic agent may be covalently or non-covalently bonded to the nanostructure. The therapeutic or diagnostic agent may be present on the outer surface of the nanostructure or inside the nanostructure. The therapeutic or diagnostic agent may be an antibiotic, a toxin, a drug such as a chemotherapeutic drug, a polynucleotide, a polypeptide or an antibody. The coated nanostructure, such as a coated carbon nanostructure may be used as an imaging or contrast agent, for example in magnetic resonance imaging or in thermo-acoustic or photo-acoustic tomography.

Preferably, a coated nanostructure of the invention may be used to deliver a therapeutic agent, such as a drug, to a cell in vivo. The cell may be a cancer cell. The coated nanostructure may be used to treat a cancer or tumour. Delivery to a cell may also be carried out in vitro or ex vivo. The coated nanostructure may be used to deliver an agent such as a chemotherapeutic drug, anticancer drug or toxin to a cancer cell or tumour. The chemotherapeutic drug, anticancer drug or toxin may be non-covalently bonded to the twist-strained double-stranded circular DNA. The delivery of the therapeutic agent to the cell may comprise relaxation of the twist-strained double-stranded circular DNA. The delivery of the therapeutic agent may comprise removal of the twist-strained double-stranded circular DNA from the nanostructure.

The coated nanostructures may also be used for tissue engineering or in nanodevices for medicine or surgery. The coated nanostructures may be used for vaccine delivery or in cell therapy, such as in cell transplantation or in stem cell therapy. The coated nanostructures may be used in nanodermatology or in nanodentistry.

Further Applications

The coated nanostructures of the invention may be used in diagnostic applications, such as in chemical or biological sensors, chips or other nanodiagnostic devices. The coated nanostructures may also be used in energy applications such as in solar cells, photovoltaic device, batteries and ultra-capacitors. A solar cell may comprise a mixture of coated nanostructures of the invention, including coated carbon nanotubes and coated carbon fullerenes. The coated nanostructures may be used for gas storage, in particular storage of hydrogen gas as described above.

The coated nanostructures may be used in microelectromechanical systems, nanoelectronmechanical systems and in nanoelectronics. The coated nanostructures of the invention may find further applications in food packaging, textiles and nanofabrics, in ceramic engineering and as structural composite materials. The coated nanostructures may be used for cleaning or in environmental nanotechnology for treatment of air or water pollution. Examples of products in which the coated nanostructures may be comprised include flat-panel displays, antifouling paint, radar-absorbing coatings, conductive plastics and atomic force microscope tips.

In all the above medical and non-medical applications, the coated nanostructures of the invention may be used directly in coated form. Alternatively, coated nanostructures of the invention may be uncoated using the method of uncoating of the invention and then used in uncoated form in the applications described.

### EXAMPLES

### Example 1

### Preparation of DNA and SWCNTs

### Preparation of Relaxed DNA

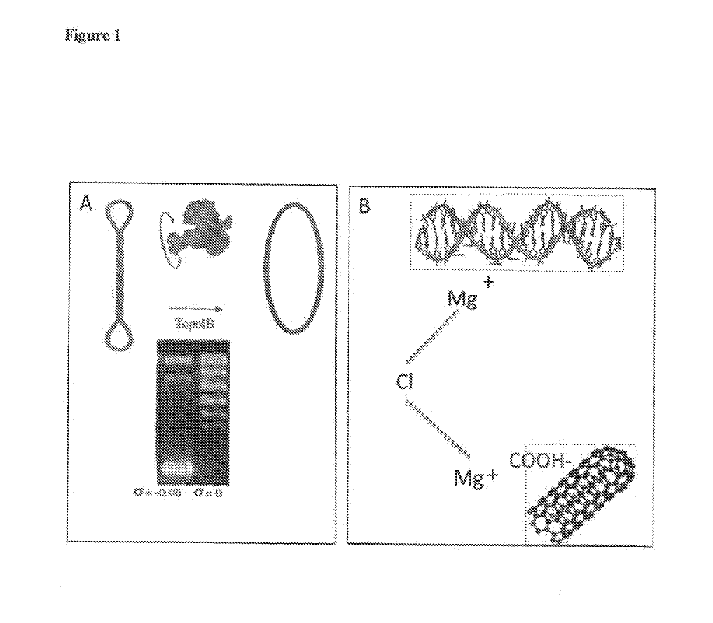

Commercially available relaxed pBR322 DNA (TG2037-1 TopoGen) was used. Alternatively, a native negatively supercoiled DNA was incubated with DNA Topoisomerase I from Vaccinia Virus (epicenterVT710500) to form relaxed DNA. Incubation buffer: Tris-HCl pH8 10 mM, 50 mM NaCl, 1 mM DTT, 5 mM MgCl2 (Trigueros et al, Journal of Molecular Biology, vol. 335, pp 723-731 (2003). Sample incubation was for 30 minutes at room temperature. The reaction was stopped by adding 5 mM EDTA, 0.1% SDS and 0.1 mg/ml Proteinase K. The relaxed DNA was ethanol precipitated and resuspended in the desired volume of Milli-Q H2O. DNA was present at 1 mg/ml with sizes in the range 3 kb-5 kb (see FIG. 1).

### Preparation of SWCNT

4 mg of SWCNTs (Sigma-Aldrich) were added to a 4 ml mixture of concentrated sulphuric acid (99.999%) and nitric acid (70%) (3:1, H₂SO₄:HNO₃). The SWCNT acid solution was then sonicated in a bath sonicator at 60 degrees centigrade for 4.5 hours. This resulted in SWCNT lengths mostly in the range 10-50 nm. To increase the length of the SWCNTs, the time of acid treatment was reduced. A treatment time of 1.5 hours produced SWCNTs having lengths in the desired range of 100-500 nm. The acid treatment resulted in the addition of carboxylic acid groups to the surface and open ends of the SWCNTs (Liu et al, Science, vol. 280, pp 1253-1256 (1998), Saitoa et al (Physica B: Condensed Matter vol 323, pp 280-283 (2002).

To remove the SWCNTs from the acid, the solution was filtered using a three-piece glass funnel set and vacuum pump. Filters with 0.47-μm-pore diameter, made from hydrophilised PTFE were used because of their suitability for use with strong acids. To remove excess acid and unwanted products of the reaction, the SWCNT coated filter was washed with 2 litres of Milli-Q water, while remaining under vacuum filtration. The pH of the SWCNTs was checked with an indicator strip to ensure that it was as close to neutral as possible. The SWCNT coated filter was put in a glass vial containing 8 ml of Milli-Q water and bath sonicated to remove the SWCNTs. The filter was then removed from the solution.

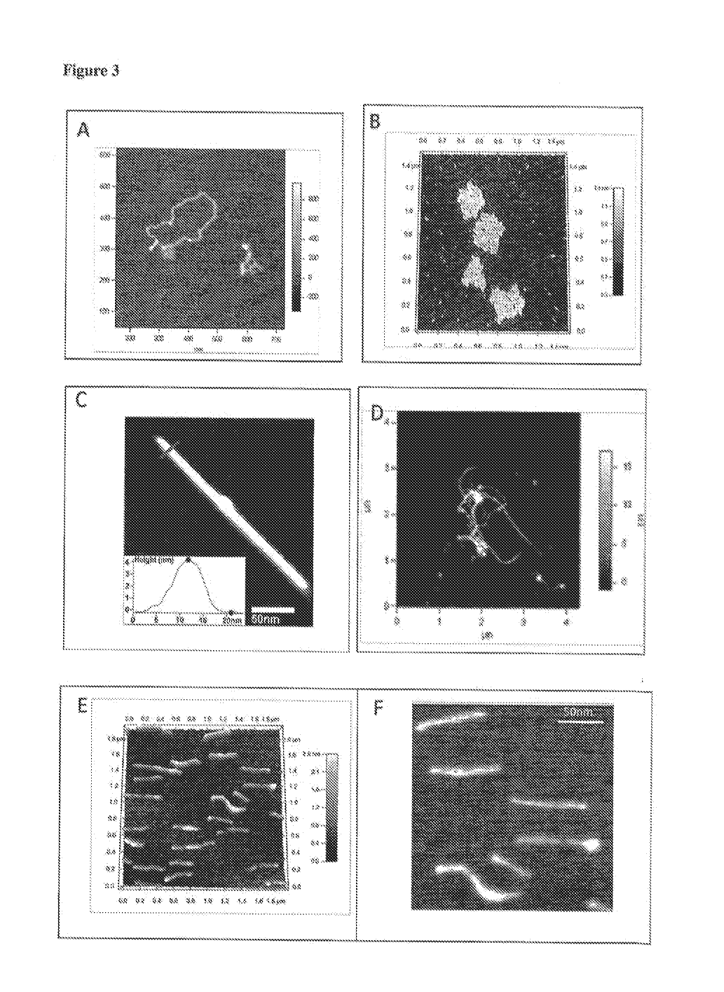

To remove small carbonaceous debris created during the acid treatment (not removed by washing) multiple precipitation steps were implemented. The solution was centrifuged at 13000 rpm for 10 minutes. The SWCNTs precipitated at the bottom of the solution, leaving the small debris in the supernatant, which was removed. The precipitated pellet was resuspended in a glass vial containing 4 ml of Milli-Q water by means of bath sonication for 10 minutes. This precipitation process was repeated four times (see FIG. 3 Panels C and D).

### Example 2

### Coating of SWCNTs with Twist-Strained Double-Stranded Circular DNA

SWCNTs produced in accordance with Example 1 at 70 microgram/ml were pre-incubated at 100 mM MgCl₂ for 10 minutes at room temperatures.

0.5 ul of 1 microgram/ml relaxed double-stranded circular DNA produced in accordance with Example 1 was then incubated together with 50 ul of the SWCNTs in a final reaction volume of 100 microlitres. Incubations were carried out in 15 mM NaCl, 70 mM MgCl₂, pH 7.5 at 4 degrees centigrade (on ice) for a minimum of 3 hours to overnight.

The above incubation conditions resulted in a yield of 60-70% of coated carbon nanotubes.

### Example 3

### Separation of Coated Carbon Nanotubes

Uncoated SWCNTs were removed from a sample incubated in accordance with Example 2 by centrifugation at 5000 rpm for 3 min in a Biofuge pico Heraeus centrifuge. The supernatant comprised soluble SWCNT coated with DNA and free DNA. The uncoated SWCNTs formed a dark pellet.

Free DNA was removed from solution by selective purification of coated SWCNTs using a modified filter binding protocol (Osheroff, DNA Topoisomerase Protocols, Vol. 95, Humana Press (1999). The filter used was a 10401191 BA85 0.45 μm Protran-Nitrocellulose (NC) blotting membrane. This nitrocellulose membrane has a binding capacity of 80 to 150 μg/cm2. Small or free DNA molecules are not retained on the filter. Consequently, passing the sample through the filter allows for removal of free DNA and other small by-pass products of the reaction.

Filtration was carried out in a microcentrifuge tube. The filter was pre-equilibrated by adding to the top of the filter (located on the top of the tube) 100 microlitres of 100 mM MgCl₂ and leaving for 5 min at room temperature to ensure full filter equilibration. The tube was then centrifuged at 3000 rpm for 2 min to remove the buffer without allowing the filter to dry.

A sample containing coated SWCNTs and free DNA was then applied to the top of the filter and incubated for 10 min at room temperature. The tube was then centrifuged at 3000 rpm for 2 min.

The filter was then washed by adding 100 microlitres of 100 mM MgCl₂ for 5 min at room temperature and centrifuging at 3000 rpm for 2 min.

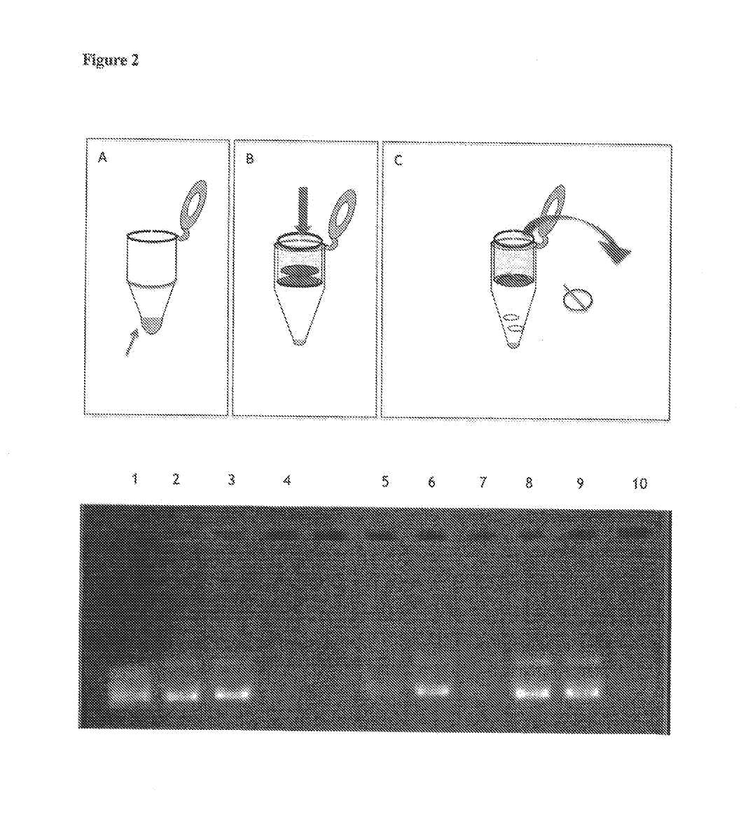

The above steps allowed for removal of free DNA which passed through the filter. 50 microlitres of 100 mM MgCl₂ was then added to the top of the filter and incubated for 5 min at room temperature. The resulting sample containing coated SWCNTs was then collected from the top of the filter. The filter binding method and data for purification of coated SWCNTs is shown in FIG. 2.

### Example 4

### Uncoating of DNA from Carbon Nanotubes

The reversibility of the interaction between SWCNTs and DNA was achieved by various methods, which produced an alteration of the DNA topology, unwinding of the double helix, and detachment of the DNA from the SWCNTs. The formation of free SWCNT form could be easily identified by the formation of a dark-black precipitate.

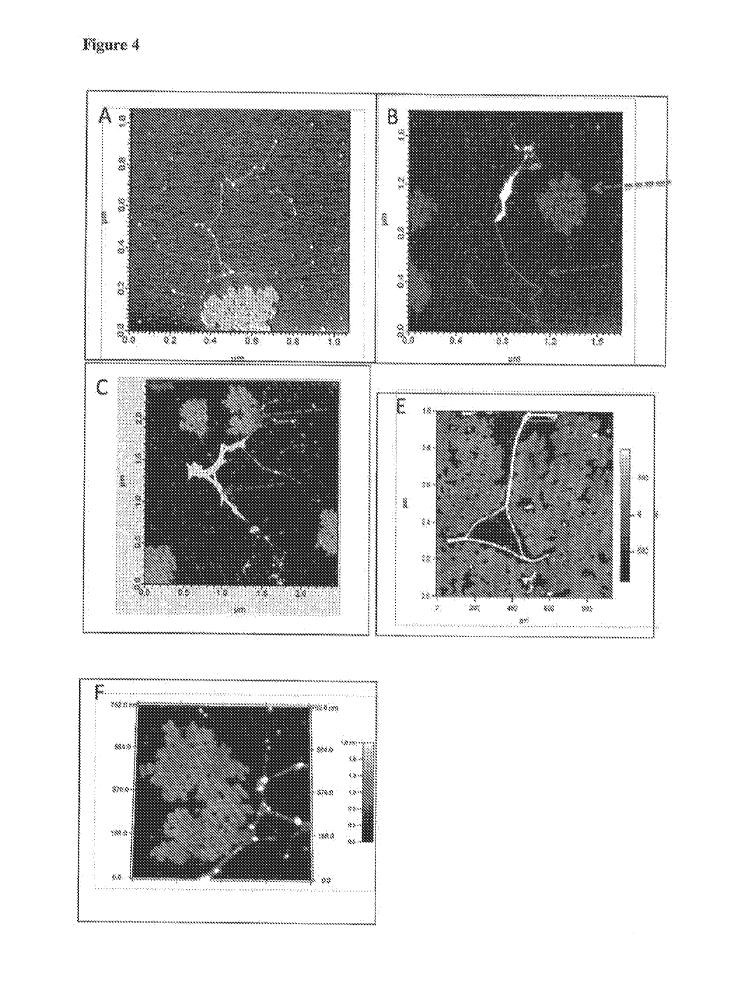

Reversibility was achieved by sample dilution, dialyzing the sample to a final concentration of 15 mM MgCl₂ or by heating the sample to 45 degrees centigrade or higher. The uncoating of the DNA was visualised by atomic force microscopy with results shown in FIG. 4.

### Example 5

### Stability of Coated Carbon Nanotubes

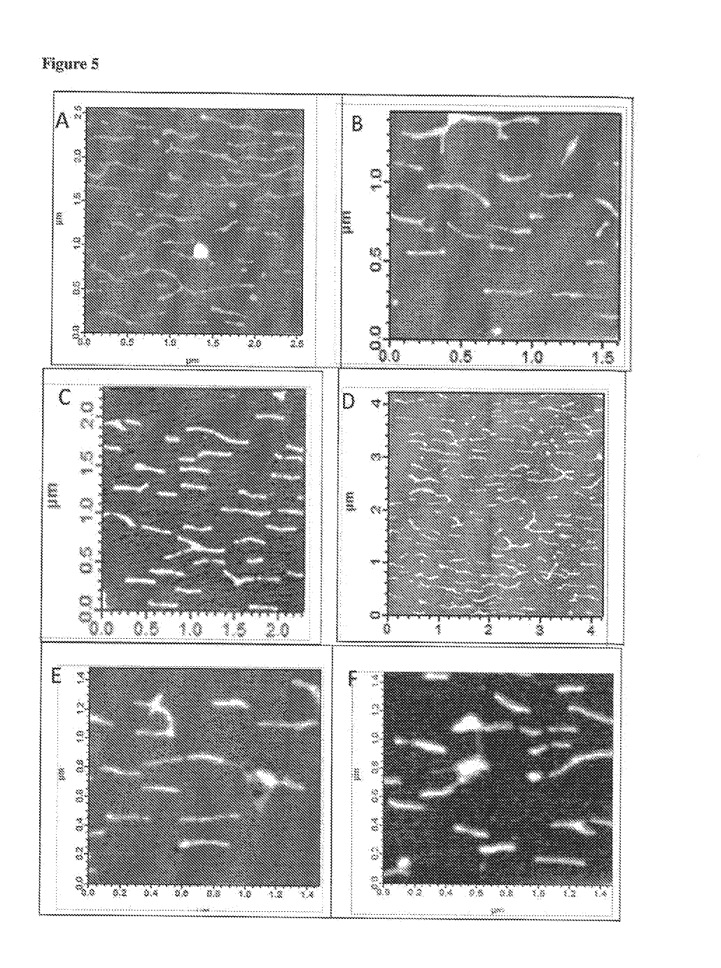

Investigations were carried out to determine the stability of coated carbon nanotubes under various conditions. Samples incubated under various conditions and for specified time periods were visualised by atomic force microscopy with results shown in FIG. 5.

The results showed that the coated carbon nanotubes remained stable at temperatures below 45 degrees centigrade and in physiological mediums (for more than 3 months) in Bacteria growth medium “LB (L-Broth or Luria Bertani) Medium”, Yeast growth Medium YPD (yeast extract, peptone, dextrose; also called YEPD media), Fetal Bovine Serum.

What is claimed is:

1-23. (canceled)

24. A nanostructure coated with a twist-strained double-stranded circular deoxyribonucleic acid (DNA).

25. The nanostructure of claim 24, wherein the nanostructure is a carbon nanostructure.

26. The nanostructure according to claim 25, which is a single wall carbon nanotube.

27. The nanostructure according to claim 26, which is 100-700 nanometers in length.

28. A method for
(I) coating a nanostructure with a twist-strained double-stranded circular DNA, comprising incubating a nanostructure with a relaxed double-stranded circular DNA under conditions promoting a change in topology of said DNA to twist-strained double-stranded circular DNA, to thereby coat the nanostructure;
(II) removing a twist-strained double-stranded circular DNA from a nanostructure coated with twist-strained double-stranded circular DNA, comprising incubating said nanostructure under conditions to relax the twist-strained double-stranded circular DNA; or
(III) sorting nanostructures by size, comprising separating nanostructures that are coated with twist-strained double-stranded circular DNA.

29. The method according to claim 28, wherein in (I) said conditions comprise the presence of divalent cations.

30. The method according to claim 29, wherein said conditions comprise the presence of Mg²⁺.

31. The method according to claim 30, wherein said conditions comprise a Mg²⁺ concentration of about 60 to about 80 mM.

32. The method according to claim 28, wherein in (I) said conditions comprise a temperature of about 0 degrees centigrade to about 10 degrees centigrade.

33. The method according to claim 28 which further comprises in (I) separating a nanostructure that is coated with twist-strained double-stranded circular DNA from free DNA.

34. The method according to claim 28 which further comprises in (I) separating a nanostructures that is coated with twist-strained double-stranded circular DNA from uncoated nanostructures.

35. The method according to claim 28, which in (II) comprises reducing the concentration of divalent cations.

36. The method according to claim 35, wherein the conditions comprise a Mg²⁺ concentration of less than about 30 mM.

37. The method according to claim 28, which in (II) comprises increasing the temperature.

38. The method according to claim 37, wherein the temperature is increased to about 45 degrees centigrade or higher.

39. The method according to claim 28, which in (III) comprises sorting nanostructures which are 100-700 nm in length.

40. The method according to claim 28 wherein the nanostructure is a carbon nanostructure.

41. A method for:
(I) treatment of the human or animal body by surgery or therapy or diagnosis practised on the human or animal body, using a nanostructure coated with a twist-strained double-stranded circular DNA;
(II) delivering a diagnostic or therapeutic agent, using a nanostructure coated with a twist-strained double-stranded circular DNA;
(III) delivering a therapeutic agent to a subject in need thereof comprising administering an effective amount of said therapeutic agent by delivery of a nanostructure coated with a double-stranded circular DNA; and
(IV) using a nanostructure coated with a twist strained double-stranded circular DNA in a chemical or biological sensor, a solar cell, photovoltaic device, battery, ultra-capacitor, or in a microelectromechanical system, nanoelectromechanical system or in a nanoelectronic device.
